(19) United States
(12) Patent Application Publication
(10) Pub. No.: US 2008/0317682 A1
(43) Pub. Date: Dec. 25, 2008

(54) Tooth Bleaching Agent

(75) Inventors: Alexander Karl Huwig, Buchs (CH); Gabriele David, Lindau (DE); Antonio Mata, Lisbon (PT); Duarte Marques, Lisbon (PT)

Correspondence Address: BOND, SCHOENECK & KING, PLLC, ONE LINCOLN CENTER, SYRACUSE, NY, 13202-1355, US

(73) Assignee: IVOCLAR VIVADENT AG, Schaan (LI)

(21) Appl. No.: 12/183,477

(22) Filed: Jul. 31, 2008

Related U.S. Application Data
(62) Division of application No. 11/492,940, filed on Jul. 26, 2006.

(30) Foreign Application Priority Data
Apr. 28, 2006 (EP) 06113321.1

(51) Int. Cl. A61K 8/22; A61K 8/18; A61Q 11/00

(52) U.S. Cl. 424/53; 424/49

(57) ABSTRACT

A bleaching agent for brightening teeth that is comprised of a solvent, a pharmaceutically acceptable oxidation agent, and a varnish former. A kit that includes a bleaching agent and lip and cheek retractor.

### CROSS REFERENCE TO RELATED APPLICATIONS

The present application is a divisional application of Applicant's co-pending U.S. patent application Ser. No. 11/492,940, filed on Jul. 26, 2006, which claims priority, pursuant to 35 U.S.C. §119, to European Patent Application No. 06113321.1 filed Apr. 28, 2006, the entire contents of which are incorporated herein by reference.

### FIELD OF THE INVENTION

The present invention relates to a bleaching agent for brightening teeth.

### BACKGROUND

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art.

Numerous foods and semi-luxury products, such as tea, coffee, red wine and tobacco, can lead to discolorations of the teeth. Certain medicinal products, such as for example tetracycline or the absorption of large quantities of fluoride can also cause discolorations of the teeth. Because the mouth, as an eye-catching feature in the centre of the face, largely determines the outward appearance such discolorations are often perceived to be a drawback, and agents and methods for their removal are attracting increasing interest.

A frequently used method comprises the bleaching of the teeth with concentrated hydrogen peroxide solution (30 to 37%). Heat and light are used to promote the oxidative action of the peroxide. Although brightening of the teeth can be achieved with this method, it requires a great outlay, and the use of a relatively high active ingredient concentration can cause sideeffects such as an irritation of the mucous membrane or hypersensitive teeth.

U.S. Pat. No. 4,032,627 discloses pigment-containing varnishes for concealing discolorations. These varnishes have a low mechanical load-bearing capacity and must be reapplied on an almost daily basis.

U.S. Pat. No. 5,290,566 discloses agents in gel form for brightening teeth which contain 22 to 32 wt.-% carbamide peroxide as active constituent. These gels are applied to a splint which is then fitted onto the teeth to be treated. This splint must be worn daily for at least 6 hours until the desired degree of brightening has been achieved.

Compositions in paste form which contain abrasives in addition to carbamide peroxide are known from U.S. Pat. No. 5,171,564.

U.S. Pat. No. 5,425,953 discloses liquid polymer compositions which, in addition to an oxidizing agent such as carbamide peroxide, contain a water soluble cellulose polymer and preferably also a stabilizer such as calcium disodium edetate. The compositions are applied one to three times daily to the teeth where, after the evaporation of the solvent, they form a film which releases the oxidation agent and decomposes within about 1 hour.

Bleaching agents in varnish form based on carbamide peroxide’ which are intended to adhere to the surface of the tooth for more than 1 hour are also known from U.S. Pat. No. 6,083,421. Preferred film-formers are polyvinyl butyral, coumaron resin and shellac. After drying, the varnishes have only a low mechanical stability and are easily destroyed upon contact with the tongue or the oral mucous membrane.

Finally, DE 20 2004000552 U1 describes oral compositions which, in addition to a pharmaceutically acceptable oxidation agent and solvent, contain water-insoluble alkyl cellulose as varnish former. The compositions are suitable in particular for application by the patient himself, and a twice daily application over a period of fourteen days is recommended.

As a rule, bleaching agents in varnish form are easy to handle, and their use involves relatively few discomforts for the patient. However, the disadvantage of the known methods for brightening teeth is that the bleaching action is often just average, with the result that there is a need for further improved methods for bleaching teeth.

### SUMMARY

Bleaching agents can be used which contain the following components:
(i) 50-96 wt.-% solvent,
(ii) 2-30 wt.-% pharmaceutically acceptable oxidation agent and
(iii) 2-30 wt.-% varnish former.

It was found that the use of the bleaching agent in combination with a lip and cheek retractor leads to a surprisingly high degree of brightening of the teeth if the lip and cheek retractor is not only used when applying the bleaching agent, but is left in the mouth during the whole action time of the bleaching agent. Even when using bleaching varnishes with low levels of oxidation agent, high degrees of brightening could be achieved. Moreover, the work space created by the lip and cheek retractor facilitates the targeted brightening of individual teeth and the removal of the bleaching agent.

### DETAILED DESCRIPTION

Surprisingly it was found that a method according to the invention makes possible a brightening of the teeth by up to 10 shades in only one to two sessions. This is astonishing inasmuch this brightening is also achieved with known bleaching agents that produce only a slight brightening when used with known treatment plans. The brightening of the teeth is for purely cosmetic purposes and does not involve any periodontis- or cariesprophylactic effect.

A brightening of at least 6, preferably at least 8 and particularly preferably at least 10 shades is preferred. To measure the brightening, firstly the starting situation is documented by ascertaining the current tooth color using a color scale such as for example the VITAPAN@ classic color key and photographing it (image with reference sample in the picture). After the method according to the invention has been carried out, the current situation is ascertained and documented again.

To guarantee an even, spot-free brightening of the tooth or teeth to be treated, the method according to the invention is preferably used on cleaned teeth, in particular on teeth that are free of plaque, Le., teeth from which hard and soft deposits and plaque residues have been removed. For this, the teeth can undergo a professional teeth cleaning carried out by the dentist or a trained dental assistant before carrying out a method according to the invention.

To avoid an irritation of soft tissue such as gingiva and oral mucous membrane by the bleaching agent used or to protect teeth that are not to be bleached, areas that are not to come into contact with the bleaching agent, in particular the gums, the gum margin and sensitive teeth, can be covered with a covering agent. The covering agent must be suitable for use in the patient's mouth. Vaseline is a preferred covering agent, but other dental greasing and insulating agents also come into consideration. In addition, the use of a cofferdam to protect gums and oral mucous membrane is preferred. The use of a covering agent prevents the bleaching agent from entering the periodontal pockets.

According to one embodiment of the method according to a invention, a lip and cheek retractor is used when carrying it out, as a result of which the lips and cheeks that come to rest against the teeth rows are held back from the teeth rows, releasing sufficient space to work. A lip and cheek expander is preferably used which has two expander frames as well as non-profiled film-like means connecting the expander frames. Such lip and cheek retractors are described, for example, in WO 03/051185 and are available under the name OptraGate® from Ivoclar Vivadent AG.

It was found that the use of the bleaching agent in combination with a lip and cheek retractor leads to a surprisingly high degree of brightening of the teeth if the lip and cheek retractor is not only used when applying the bleaching agent, but is left in the mouth during the whole action time of the bleaching agent. Even when using bleaching varnishes with low levels of oxidation agent, high degrees of brightening could be achieved. Moreover, the work space created by the lip and cheek retractor facilitates the targeted brightening of individual teeth and the removal of the bleaching agent.

It is furthermore preferred to dry the tooth surface to be bleached before application of the bleaching agent. The drying can take place, e.g., by blowing with an air jet or by dabbing with a tissue.

The application of the bleaching agent in step (a) to the tooth surface to be bleached can be carried out with an applicator, e.g., a small brush. In view of a possible precipitation of the varnish former of the oral composition upon contact with saliva, the required quantity of bleaching agent is preferably transferred from the pack into for example a small bowl and then applied to the teeth with the applicator. In this way, contact between the composition in the pack and the applicator used is avoided.

After application, in step (b) the bleaching agent is left to act on the tooth or teeth. For this, the bleaching agent is preferably allowed to dry. The drying can be accelerated by blowing with an air jet. In the case of the preferred bleaching agents, upon evaporation of the solvent a colorless to white or optionally colored varnish layer forms which bonds securely to the tooth surface and remains in the mouth for the desired time.

After the action time has elapsed, the formed varnish is removed in step (c). The bleaching agents preferred according to the invention form a varnish layer which can be peeled off like a film or removed with a wet toothbrush. But it is also possible to use an ultrasound or slowly rotating, non-cutting device to remove the varnish layer.

Steps (a) to (c) can be repeated one or more times.

Bleaching agents can be used which contain the following components:
(iv) 50-96 wt.-% solvent,
(v) 2-30 wt.-% pharmaceutically acceptable oxidation agent and
(vi) 2-30 wt.-% varnish former.

These bleaching agents are varnishes. They are therefore also called bleaching varnishes below.

Pharmaceutically acceptable oxidation agents are sodium perborate, chlorine dioxide and pharmaceutically acceptable peroxide compounds such as, e.g., organic peroxides, preferably methyl peroxide, ethyl peroxide and in particular glycerol peroxide or benzyl peroxide. Particularly preferred peroxides are hydrogen peroxide and carbamide peroxide as well as mixtures of these two substances. Carbamide peroxide is preferably used in a quantity of 4 to 20 wt.-%, particularly preferably 5 to 10 wt.-% and quite particularly preferably in a quantity of 5.3 to 6.5 wt.-%. Hydrogen peroxide is preferably used in a quantity of 2 to 20 wt.-%, particularly preferably 5.5 to 13 wt.-% and most preferably 5.5 to 12 wt.-%, measured as H202.

Hydrogen peroxide can be used in the form of a commercially available hydrogen peroxide solution in water with a H202 concentration of 34.5 to 36.5%. For higher hydrogen peroxide concentrations, a likewise commercially available aqueous H202 solution with a concentration of 48 to 70% is preferably used.

Carbamide peroxide hydrolyzes in the presence of water to form urea and hydrogen peroxide. The formed urea has the advantage that it has a pH-neutralizing effect. The released hydrogen peroxide diffuses into the tooth enamel and the dentine where it decomposes upon oxidation into water and oxygen. As a result of the diffusion of the hydrogen peroxide into the tooth enamel, the chemical equilibrium between carbamide peroxide and its decomposition products is disturbed, which results in the further release of hydrogen peroxide from the carbamide peroxide. The varnishes according to the invention ensure that the hydrogen peroxide almost completely diffuses into the tooth enamel and is not, as with conventional varnishes, largely released in saliva and soft tissue.

To be able to exploit the pH-neutralizing effect of the urea contained in the carbamide peroxide, mixtures of hydrogen peroxide and carbamide peroxide can also be used. For this, both peroxide compounds are preferably used in a quantity of up to 20 wt-%. Hydrogen peroxide is particularly preferably used in a quantity of 4 to 20 wt.-% and carbamide peroxide in a quantity of 5 to 10 wt.-%.

Compositions used include varnishes which preferably display a viscosity of 5 to 20,000 mPas, particularly preferably 100 to 5,000 mPas and quite particularly preferably 1,000 to 5,000 mPas. Unless otherwise indicated, all the viscosities given here are values which have been determined with a rotational viscometer (measured at 23.0° C., shearing rate 1 00S-1, conical plate measurement system, cone diameter 20 mm, aperture angle 2.5°, gap 70 I-Im).

The viscosity is set by adding the varnish former. Preferred varnish formers are cellulose-based, in particular alkyl cellulose-based, varnish formers.

A water-insoluble alkyl cellulose is preferably used, i.e., alkyl cellulose with a solubility of at most 0.1 g alkyl cellulose per 100 g water at room temperature. Ethyl cellulose is particularly preferred, in particular ethyl cellulose with an average degree of substitution (OS) of>1.5, particularly preferably>2.0, in particular 2.0 to 2.6, and most preferably 2.3 to 2.4. In cellulose, each glucose unit has three free hydroxyl groups. If all three hydroxyl groups of all the glucose units of the cellulose are substituted by alkoxy groups, the degree of substitution (OS) is 3. If only some of the hydroxyl groups of the glucose units are substituted by alkoxy groups, the degree of substitution is correspondingly lower. The degree of substitution OS gives the average number of substituted hydroxyl groups per linked glucose unit. Ethyl cellulose which has an ethoxyl content of 45 to 50% is quite particularly preferred.

The required quantity of varnish former depends on the type of varnish former and is preferably such that the varnish has a viscosity within the ranges given above. In the case of the alkyl cellulose preferably used as varnish former, the quantity depends on among other things its chain length and molecular weight and thus on the viscosity formed in the solution. Alkyl cellulose is preferably used in a quantity of 2 to 30 wt.-%, particularly preferably 5 to 20 wt.-%, quite particularly preferably 6 to 12 wt.-%. A person skilled in the art can directly ascertain on the basis of his expert knowledge the quantities of other varnish formers required to set the viscosity of the bleaching varnish.

Preferred solvents are diethyl ether, acetone, esters, such as for example ethyl acetate, and alcohols, such as isopropanol and in particular ethanol. Particularly preferred solvents are ethyl acetate and ethanol. Mixtures of one or more of these solvents can be used. In addition, the solvent can contain up to 30 wt.-% water relative to the total solvent quantity, i.e. to the quantity of water and further solvent. When using carbamide peroxide, the water content is preferably less than 12 wt.-%, particularly preferably less than 2 wt.-%, anhydrous compositions being most preferred.

According to one embodiment, the compositions, in addition to the named components, also contain a pharmaceutically acceptable acid, preferably phosphoric acid (H3P04), pyrophosphoric acid (H4P207) and in particular citric acid. The acid is preferably used in a quantity of 0.05 to 5.0 wt.-%, in particular 0.5 to 5 wt.-%, particularly preferably 0.3 to 0.6 wt.-%. Unless otherwise indicated, all percentages relate to the overall mass of the composition. As a result of the acid, a firm and even bonding of the varnish to the tooth surface is ensured without however damaging the tooth.

The compositions may also contain as a further optional component dexpanthenol, preferably in a quantity of 0.1 to 1.0 wt.-%, particularly preferably 0.2 to 0.5 wt.-%. Dexpanthenol is a provitamin that counteracts irritations and inflammations of the oral mucous membrane which can be triggered by the peroxide compound, the solvent or the acid. Compositions containing dexpanthenol are therefore usually better tolerated than customary agents.

In addition, further components, such as aromatics, flavorings and/or sweeteners can be added to the composition. These components are each preferably used in a quantity of 0.01 to 0.5 wt.-%, particularly preferably 0.02 to 0.2 wt.-% and quite particularly preferably 0.03 to 0.06 wt. %.

Preferred aromatics and flavorings are methyl salicylate, sassafras oil, clove oil, salvia oil, eucalyptus oil, marjoram oil, menthol and preferably mint oil, i.e., peppermint and/or spearmint oil. Particularly preferred are synthetic mint oils and aromas, in particular, the oils and aromas marketed under the name OPTAMIN-r® by Symrise GmbH. Other suitable aromas are strawberry, raspberry, banana, cherry, caramel, pear, apple, lemon and peach aroma as well as orange oil and grapefruit aroma.

Sodium saccharin, sodium cylamate, xylitol, aspartame and similar are particularly suitable as sweeteners. Sodium saccharin is particularly preferred.

The compositions can be colorless to white varnishes, which according to a certain embodiments are neutral, i.e., have a pH in the range from 5 to 8, preferably 6.5 to 7.5, measured on an aqueous dispersion of the composition (1 to 25 wt.-%). The varnishes do not drip, do not pull threads when applied and can be applied precisely with a brush. They dry within approximately 1 minute.

Acid-containing compositions preferably have a pH of 1 to 6, particularly preferably 2 to 4 and quite particularly preferably 2 to 3.

According to one embodiment, the compositions can also contain colorants which do not have a negative influence on the bleaching effect, for example, carmine and carthamin. By using colored compositions, it can be more easily ensured that the tooth surface is fully wetted. The compositions preferably contain no abrasives.

Preferred compositions contain the following components:
(i) 50.0 to 96.0 wt.-%, preferably 60 to 90 wt.-%, in particular 80 to 88 wt.-%, quite particularly preferably 84 to 86 wt.-% solvent, preferably ethanol,
(ii) 2 to 20 wt.-% oxidation agent, in particular 5.3 to 6.5 wt.-% carbamide peroxide or 6 to 20 wt.-% hydrogen peroxide,
(iii) 2 to 30 wt.-%, preferably 8.0 to 15.0 wt.-%, in particular 9 to 12 wt.-% varnish formers, preferably alkyl cellulose, particularly preferably ethyl cellulose,
(iv) 0 to 5.0 wt.-%, in particular 0.5 to 5 wt.-%, particularly preferably 0.3 to 0.6 wt.-% acid, preferably citric acid,
(v) 0 to 1.0 wt.-%, in particular 0.2 to 0.5 wt.-% dexpanthenol,
(vi) 0 to 0.5 wt.-%, in particular 0 to 0.2 wt.-%, particularly preferably 0.01 to 0.05 wt.-% flavoring and/or aromatic, in particular mint oil or mint aroma.

A particularly preferred bleaching agent based on hydrogen peroxide has the following composition:
(i) 55 to 80 wt.-%, in particular 62 to 80 wt.-% ethanol,
(ii) 5.5 to 20.0 wt.-%, in particular 6 to 20 wt.-% hydrogen peroxide,
(iii) 5.5 to 12.5 wt.-%, in particular 8.5 to 9.5 wt.-% ethyl cellulose,
(iv) 0 to 28 wt.-%, in particular 6 to 12 wt.-% water,
(v) 0.2 to 0.5 wt.-% dexpanthenol and
(vi) 0.01 to 0.06 wt.-% aroma.

Here, the water content relates to the overall mass of the composition, wherein however in addition the maximum water content defined above is also to be observed relative to the total quantity of solvent.
A quite particularly preferred bleaching agent based on carbamide peroxide has the following composition:
(i) 79 to 85 wt.-% ethanol,
(ii) 5.3 to 6.5 wt.-% carbamide peroxide,
(iii) 8.5 to 9.5 wt.-% ethyl cellulose,
(iv) 0 or 0.3 to 0.6 wt.-% citric acid,
(v) 0.2 to 0.5 wt.-% dexpanthenol and
(vi) 0.01 to 0.06 wt.-% aroma.

The compositions used according to the invention are prepared by mixing the components together in the desired quantities until a homogeneous, spreadable solution with the desired viscosity is obtained.

A subject of the invention is also the use of a pharmaceutically acceptable oxidation agent for the preparation of an agent for brightening teeth according to the treatment plan in which:
(a) a bleaching agent is applied to the surface of the tooth or teeth to be bleached,
(b) the bleaching agent is left to act on the tooth for a period of 3 to 30 minutes,
(c) the bleaching agent is removed from the tooth surface,
(d) steps (a) to (c) are repeated at least once.

Preferred versions of the treatment plan follow from the above description.

The present invention may also include a kit for brightening teeth which contains the materials necessary for carrying out the method according to the invention, in particular
(A) a bleaching agent,
(B) a lip and cheek retractor.

In addition, the kit may also contain (C) an applicator for applying the bleaching agent to the tooth or teeth and/or (0) a covering agent. Preferred embodiments of the kit according to the invention are those in which the components (A) to (0) are characterized by the advantageous features described above.

The kit preferably contains one or more brushes as applicator. Furthermore, the pack can also contain (E) a vessel, preferably a small bowl, for receiving the quantity of the bleaching agent required for one application. The kit preferably also contains (F) instructions for use which describe how to carry out the method according to the invention.

The invention is described in further detail below with reference to embodiment examples, which are to be considered illustrative and not necessarily limiting the scope of the present invention.

### EXAMPLES

### Examples 1-6

### Preparation of Oral Compositions

Bleaching varnishes with the compositions given in Table 1 were prepared. For this, ethanol was poured into a beaker and ethyl cellulose added to it. The mixture was stirred until a homogeneous solution was present. Carbamide peroxide or hydrogen peroxide, dexpanthenol and aroma were then added successively and the mixture stirred evenly. Colorless to white varnishes were obtained.

The following bleaching agents were prepared according to the methods described in Examples 1 and 8.

TABLE 1
Composition in wt.-% of the prepared oral compositions

| | Example | | | | | |
|---|---|---|---|---|---|---|
| Component | 1^{B} | 2^{b} | 3 | 4 | 5 | 6 |
| Ethanol^{c} | 84.7 | 70.6 | 78.67 | 72.67 | 66.67 | 62.10 |
| | 5.9 | 6.0 | 6.00 | 9.00 | 12.00 | 20.00 |
| Oxidation agent | (CP) | (HP) | (HP)^{d} | (HP)^{d} | (HP)^{d} | (HP)^{e} |
| Water | | 14.0 | 6.00 | 9.00 | 12.00 | 8.57 |
| Ethyl cellulose^{f} | 9.0 | 9.0 | 9.00 | 9.00 | 9.00 | 9.00 |
| Dexpanthenol | 0.3 | 0.3 | 0.30 | 0.30 | 0.30 | 0.30 |
| Aroma^{g} | 0.1 | 0.1 | 0.03 | 0.03 | 0.03 | 0.03 |

(CP): contains carbamide peroxide as oxidation agent
(HP): contains hydrogen peroxide as oxidation agent
^{a}according to DE 20 2004000552 U1
^{b}according to DE 20 2004000552 U1
^{c}high-purity analytical-grade ethanol
^{d}using a 50% hydrogen peroxide solution for preparation
^{e}using a 70% hydrogen peroxide solution for preparation
^{f}ethyl cellulose EC 100
^{g}Optamint ® crisped mint

### Example 7

### Bleaching of Teeth

The tests were carried out on a total of approximately 380 volunteer subjects.

A general medical history was recorded for all subjects and dental findings compiled. A professional teeth cleaning was carried out on all subjects before the start of treatment. Only after the teeth cleaning was the starting situation documented and the current tooth color determined using the Vita color scale (VITAPAN® classic) and photographed (frontal overview image with Vita reference sample in the picture).

The subjects were then divided into groups, each with at least 10 participants.

In the case of a first group of people (Group 1) the treatment with the oxidation agent-containing bleaching agent from Example 3 was carried out according to the invention. Before each application of varnish, the gums and hypersensitive teeth were covered with Vaseline. The surfaces of the teeth to be bleached were then dried with an air blower. To make it easier to reach the teeth to be bleached, a lip and cheek retractor was used according to WO 03/051185 (OptraGate˜\Ivoclar Vivadent).

An even layer of the varnish was applied to the teeth surfaces with a brush. After the application, the lip and cheek retractor was left in the patient's mouth for the total action time of the varnish. The patient was asked not to touch the teeth with the tongue for the duration of the action time. After the action time had expired, the varnish was removed with a nylon brush and water spray, and the mucous membrane checked for irritations. Only then was the lip and cheek retractor removed.

The people were subjected to 6 bleaching agent applications of 10 minutes each over two sessions. The total action time of the bleaching varnish was thus 60 minutes per session. The sessions were carried out at intervals of one week.

The current situation was determined at each session. For this, the current tooth color was determined according to the Vita color scale and photographed. Already after the first session, a brightening by on average 6.6 shades was observed (standard deviation 2.01, N=20). In the second session, a further brightening by on average 3.06 shades was achieved (standard deviation 1.8, N=16). After only two sessions, a brightening of on average just under 10 shades according to the Vita color scale was thus achieved.

The second group (Group 2) was treated with a varnish that did not contain an oxidation agent, but was identical in appearance to the oxidation agent-containing compositions (placebo). The treatment took place as described for Group (1). With this group, a decrease in brightness of 0.4 shades according to the Vita color scale was found after the first session, a brightening of 0.8 shades after the second session, i.e., a brightening of 0.4 shades compared with the starting situation. These values are not significant.

In contrast, the treatment with the oxidation agent-containing composition from Example 1 produced a brightening of on average only 1.6 brightness stages after a customary treatment plan (DE 20 2004 000 552 U1, Example 7). The treatment was carried out over a period of two weeks and comprised a total of 14 bleaching agent applications of 15 minutes each (total action time: 210 minutes). The total action time was thus approximately double that of Group 1, and yet the achieved brightening turned out to be much less.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. A kit for brightening teeth which comprises:
(A) a bleaching agent, the bleaching agent comprising:
(i) 50-96 wt.-% solvent,
(ii) 2-30 wt.-% pharmaceutically acceptable oxidation agent and
(iii) 2-30 wt.-% varnish former, and
(B) a lip and cheek retractor.

2. The kit according to claim 1, further comprising (C) an applicator for applying the bleaching agent to the tooth or teeth.

3. The kit according to claim 2, further comprising (0) a covering agent.

4. A bleaching agent comprising:
(i) 50-96 wt.-% solvent;
(ii) 2-30 wt.-% pharmaceutically acceptable oxidation agent; and
(iii) 2-30 wt.-% varnish former.

5. The bleaching agent according to claim 4, wherein the solvent comprises alcohol, ethanol, isopropanol, diethyl ether, esters, ethyl acetate, and/or acetone.

6. The bleaching agent according to claim 4, wherein the pharmaceutically acceptable oxidation agent comprises a peroxide.

7. The bleaching agent according to claim 4, wherein the bleaching agent has a viscosity of 5 to 20,000 mPas.

8. The bleaching agent according to claim 4, the varnish former comprises alkyl cellulose.

9. The bleaching agent according to claim 8, wherein the varnish former comprises an alkyl cellulose with a water solubility of at most 0.1 g in 100 g water at room temperature.

10. The bleaching agent according to claim 4, wherein the bleaching agent further comprises 0.1 to 1.0 wt.-% dexpanthenol.

11. The bleaching agent of claim 4 comprising:
(i) 60-90 wt.-% solvent;
(ii) 5.3-6.5 wt.-% carbamide peroxide;
(iii) 8-15.0 wt.-% varnish former;
(iv) 0.5-5.0 wt.-% citric acid;
(v) 0.2-0.5 wt.-% dexpanthenol; and
(vi) 0-0.2 wt.-% flavoring and/or aromatic.

12. The bleaching agent of claim 4 comprising:
(i) 80-88 wt.-% solvent;
(ii) 6.0-20.0 wt.-% hydrogen peroxide;
(iii) 8.0-15.0 wt.-% varnish former;
(iv) 0.3-0.6 wt.-% citric acid;
(v) 0.2-0.5 wt.-% dexpanthenol; and
(vi) 0.01-0.05 wt.-% flavoring and/or aromatic.

13. The bleaching agent of claim 4 comprising:
(i) 84-86 wt.-% solvent;
(ii) 6.0-20.0 wt.-% hydrogen peroxide;
(iii) 9.0-12.0 wt.-% varnish former;
(iv) 0.3-0.6 wt.-% citric acid;
(v) 0.2-0.5 wt.-% dexpanthenol; and
(vi) 0.01-0.05 wt.-% flavoring and/or aromatic.

14. The bleaching agent of claim 4, comprising:
(i) 55 to 80 wt.-% ethanol;
(ii) 5.5 to 20.0 wt.-% hydrogen peroxide;
(iii) 5.5 to 12.5 wt.-% ethyl cellulose;
(iv) 0 to 28 wt.-% water;
(v) 0.2 to 0.5 wt.-% dexpanthenol; and
(vi) 0.01 to 0.06 wt.-% aroma.

15. The bleaching agent of claim 4, comprising:
(i) 62 to 80 wt.-% ethanol;
(ii) 6 to 20 wt.-% hydrogen peroxide;
(iii) 8.5 to 9.5 wt.-% ethyl cellulose;
(iv) 6 to 12 wt.-% water;
(v) 0.2 to 0.5 wt.-% dexpanthenol; and
(vi) 0.01 to 0.06 wt.-% aroma.

16. The bleaching agent of claim 4, comprising:
(i) 79 to 85 wt.-% ethanol;
(ii) 5.3 to 6.5 wt.-% carbamide peroxide;
(iii) 8.5 to 9.5 wt.-% ethyl cellulose;
(iv) 0 or 0.3 to 0.6 wt.-% citric acid;
(v) 0.2 to 0.5 wt.-% dexpanthenol; and
(vi) 0.01 to 0.06 wt.-% aroma.
